(19) United States
(12) Patent Application Publication
(10) Pub. No.: US 2011/0092439 A1
(43) Pub. Date: Apr. 21, 2011

(54) METHOD OF PROMOTING WOUND HEALING

(76) Inventor: Dennis R. Stewart, Los Gatos, CA (US)

(21) Appl. No.: 12/992,626

(22) PCT Filed: May 13, 2009
(86) PCT No.: PCT/US09/43854

§ 371 Date: Dec. 9, 2010

Related U.S. Application Data
(60) Provisional application No. 61/127,947, filed on May 16, 2008.

(51) Int. Cl. A61K 38/22; A61P 17/02

(52) U.S. Cl. 514/18.8

(57) ABSTRACT

The present disclosure relates to methods of administering relaxin to promote wound healing and reduce scar formation. The disclosure further contemplates the use of relaxin in reconstructive and plastic surgery.

### RELATED APPLICATIONS

This application claims the benefit under 35 USC 119(e) of U.S. Provisional Patent Application No. 61/127,947, filed May 16, 2008, which is incorporated herein by reference in its entirety for all purposes.

### FIELD

The present disclosure relates to methods for promoting wound healing and reducing scar formation. The methods described herein employ the administration of relaxin.

### BACKGROUND

Wounds often heal leaving a visible and unsightly scar, and the resulting tissue is usually weaker than the surrounding tissue because the collagen does not heal in its original orientation. Various approaches have been tried to enhance wound healing and reduce scar formation, such as the application of silicon sheets to the surface of the skin to reduce raised scar formation. Some topical creams and gels are also sold as a remedy for scars with claims of improving the scar's appearance. However, many of these approaches fail to provide the claimed solution. For example, topical creams promoted for scar reduction have not been shown to have efficacy when tested in blinded trials, as they generally do not address the collagen network that reforms following a wound.

According to market research by The Mattson Jack Group, of the total 42 million surgical procedures in the United States in 2004, 34 percent of patients were at high risk of compromised healing. This research showed that there was low satisfaction with current therapies available on the market, and a high interest in remedies that accelerate surgical healing. Thus, there is a clear market need to provide effective medication to promote the healing of wounds. Specifically, it is often desirable to increase the rate of healing in the case of acute wounds (such as penetrative injuries, burns, nerve damage or even wounds resulting from elective surgery), chronic wounds (such as diabetic, venous and decubitus ulceration) or for generally healing-compromised individuals (such as the elderly or diabetic individuals). In all these examples, wounds can severely influence an individual's quality of life, or even result in death. For example, bacterial infection of a wound site can impede the healing process, and lead to life threatening complications. Thus, it is desirable to increase the rate of healing as much as is clinically possible.

The wound healing process is a complicated series of events that begins at the moment of injury and can continue for months to years. Specifically, wound healing in adult tissues is a complicated reparative process. For example, the healing process for skin involves the recruitment of a variety of specialized cells to the site of the wound, extracellular matrix and basement membrane deposition, angiogenesis, selective protease activity and re-epithelialization (Singer and Clark, The New England Journal of Medicine, 341: 738-743, 1999).

There are three distinct phases in the wound healing process. First, in the inflammatory phase, which typically occurs from the moment a wound occurs until the first two to five days, platelets aggregate to deposit granules, promoting the deposit of fibrin and stimulating the release of growth factors. Leukocytes migrate to the wound site and begin to digest and transport debris away from the wound. During this inflammatory phase, monocytes are also converted to macrophages, which release growth factors for stimulating angiogenesis and the production of fibroblasts.

Second, in the proliferative phase, which typically occurs from two days to three weeks, granulation tissue forms and epithelialization begins. Fibroblasts, which are key cell types in this phase, proliferate and synthesize collagen to fill the wound and provide a strong matrix on which epithelial cells grow. As fibroblasts produce collagen, vascularization extends from nearby vessels to supply nutrients to the regenerating tissue. The red loops of blood vessels give the wound a granular appearance, thus the term “granulating” tissue. Epithelialization involves the migration of epithelial cells from the wound surfaces to seal the wound. Epithelial cells are driven by the need to contact cells of like type and are guided by a network of fibrin strands that function as a grid over which these cells migrate. Contractile cells called myofibroblasts appear in wounds, and aid in wound closure. These cells exhibit collagen synthesis and contractility, and are common in granulating wounds.

Third, in the remodeling phase, the final phase of wound healing which can take place from three weeks up to several years, collagen in the scar undergoes repeated degradation and re-synthesis. During this phase, the tensile strength of the newly formed skin increases.

However, as the rate of wound healing increases, there is often an associated increase in scar formation. Scarring is a consequence of the healing process in most adult animal and human tissues. Scar tissue is not identical to the tissue which it replaces, as it is usually of inferior functional quality. For example, scars in the skin are less resistant to ultraviolet radiation, and sweat glands and hair follicles do not grow back within scar tissue. The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion. Scar contractures are scars that cross joints or skin creases at right angles, and are prone to developing shortening or contracture. Scar contractures occur when the scar is not fully matured, often tend to be hypertrophic, and are typically disabling and dysfunctional. Scarring can also be ischemic or striae. Ischemic scars result from the local deficiency of blood supply. Striae scars form when skin is stretched rapidly (for instance during pregnancy, significant weight gain or adolescent growth spurts), or when skin is put under tension during the healing process, (usually near joints). This type of scar usually improves in appearance after a few years.

In contrast, normal skin consists of collagen fibers arranged in a basket-weave pattern, which contributes to both the strength and elasticity of the dermis. Thus, to achieve a smoother wound healing process, an approach that not only stimulates collagen production, but also lays down collagen in a more organized arrangement in order to reduce scar formation is desired. The disclosure addresses this unmet need.

### BRIEF SUMMARY OF THE PREFERRED EMBODIMENTS

The present disclosure generally provides methods to improve wound healing, and to reduce scar formation. Aspects of the disclosure relate to methods for promoting healing of an injury or a wound, promoting re-epithelialization of a wound, reducing scarring during healing of a wound, preventing scarring during healing of a wound, inhibiting TGF-beta collagen induced fibroblast proliferation and collagen production of a wound of the skin, and improving the appearance of disfiguration.

Wounds may result from an incision, laceration, abrasion, punctures, penetration, gunshots, stabbing, as well as facial and full-body plastic and reconstructive surgery. While various approaches, such as silicon sheets and topical creams, have attempted to enhance wound healing and reduce scar formation, many of these approaches fail to achieve the desired effect. Thus, the disclosure provides a new therapeutic approach to address this need. One advantage of this disclosure is that relaxin increases the speed of wound healing. Another advantage of the disclosure is that relaxin improves the strength of the wound site. Yet other advantages include the stimulation of anti-fibrotic actions which reduces scar formation, and the inhibition of TGF-beta collaged induced fibroblast proliferation and collagen production through administration of relaxin.

Administration of relaxin significantly enhances wound healing. For example, in rodents. systemic relaxin administration in rats is associated with upregulation of VEGF and bFGF transcripts and increased new blood vessel formation selectively at wounded sites (Unemori et al., Wound Repair and Regeneration, 8: 366-368, 2000). However, rodent wound healing mechanisms are different from pig and human wound healing mechanisms. Thus, an advantage of the present disclosure is that it provides methods to administer relaxin in the skin of animals (such as pigs) and humans, in a safe and effective way to promote wound healing and to reduce scar formation.

For each of the aforementioned methods, an animal or a human subject (including diabetic subjects) with an injury or wound (which may be an open wound, a closed wound, a cut, or a wound derived from facial plastic surgery or full-body plastic surgery) is selected, and a pharmaceutical formulation with pharmaceutically acceptable relaxin in an amount effective to promote healing of the injury or wound is administered. In certain embodiments, the injury is a cut (which may be an incision of the epidermis), or a wound (which may be open or closed). Examples of open wounds include, but are not limited to, an incision, a laceration, an abrasion, a puncture wound, a penetration wound, a gunshot wound, and a stabbing wound. Examples of closed wounds include, but are not limited to, a contusion or a hematoma. In certain embodiments, the healing of the wound, re-epithelialization, or reduction of scarring during healing is accelerated. In other embodiments, the wound is covered by a scab in whole or in part, contains active fibroblasts, or is an acute or chronic wound. In yet other embodiments, the cut is an incision of the epidermis.

In other embodiments for the aforementioned aspects of the disclosure, a wound may be derived from cosmetic surgery, such facial plastic surgery or full-body plastic surgery. Examples of facial plastic surgery include, but are not limited to, rhytidectomy, blepharoplasty, rhinoplasty, otoplasty, mentoplasty, face lift, fore head lift, brow lift, facial scar revision, facial scar removal, laser surgery, skin resurfacing, wrinkle treatment, plasma skin regeneration, facial fat grafting, skin tightening, tattoo removal and hair replacement. Examples of full-body plastic surgery include, but are not limited to, abdominoplasty, breast reduction, breast enhancement, body lift procedures, spider vein treatment, stretch mark treatment, liposuction, excess skin removal surgery, cellulite reduction treatment, body contouring, body resurfacing and body implants.

In certain embodiments, the promotion of healing a wound or cut in subjects, the promotion of re-epithelialization of a wound, the reduction or prevention of scarring during healing of a wound, or the inhibition of TGF-beta collagen induced fibroblast proliferation and collagen production of a wound occurs through the modulation of specific receptors. In particular, LGR7 and LGR8 receptors are activated through the binding of relaxin, wherein the binding triggers the production of nitric oxide (NO). In other embodiments, relaxin promotes healing of a wound or a cut by increasing vasodilation around an injury site, reducing tissue granulation at a wound site, reducing chronic inflammation at a wound site, reducing necrosis at a wound site, increasing organization of collagen at a wound site, improving wound site histology, increasing strength of a wound site and combinations thereof. The aforementioned methods further include promoting re-epithelialization of wounds of the skin, reducing scarring, preventing scarring, and inhibiting TGF-beta collagen induced fibroblast proliferation and collagen production. In certain embodiments, re-epithelialization includes, but is not limited to, increasing vasodilation around a wound site, reducing tissue granulation at a wound site. reducing chronic inflammation at a wound site, reducing necrosis at a wound site, increasing organization of collagen at a wound site, improving wound site histology, and/or increasing strength of a wound site. In other embodiments, re-epithelialization of a wound of the skin further reduces scarring, prevents scarring, and/or inhibits TGF-beta collagen induced fibroblast proliferation and collagen production.

Relaxin employed in the pharmaceutical formulations of the disclosure can be, for example, synthetic or recombinant relaxin. In one embodiment, relaxin is human relaxin. In another embodiment of the disclosure, relaxin is H2 human relaxin. In yet another embodiment, relaxin is synthetic or recombinant H2 human relaxin. Thus, the subject can be treated with a pharmaceutical formulation of synthetic or recombinant human relaxin. In another embodiment of the disclosure, the subject is treated with synthetic H2 human relaxin. In yet another embodiment, the subject is treated with recombinant H2 human relaxin.

Relaxin can be administered to the subject through a number of different routes, including, but not limited to, topically, subcutaneously, systemically, intramuscularly, sublingually, intravenously, via inhalation, via injection, via irrigation and/or via an osmotic pump (such as a multi-chamber osmotic pump system). For example, topical delivery may include, but is not limited to, lotion, gel, cream, solution and bandages (wherein relaxin is delivered on the gauze of a bandage, and when applied to a wet wound, would be released into the wound site). In certain embodiments, relaxin administered at a progressively diminishing rate. That rate can be predetermined so as to maintain a serum concentration of relaxin from about 0.5 to about 500 ng/mL, more preferably from about 0.5 to about 300 ng/mL, and most preferably from about 3 to about 75 ng/mL. A possible range is about 1 to about 50 ng/mL, wherein a preferred serum concentration is 20 ng/mL. In other embodiments, relaxin is administered in an amount ranging from about 10 to 1000 μg/kg of subject body weight per day. In yet another embodiment, the dosages of relaxin are 10, 30, 100 and 250 μg/kg/day. In yet another embodiment, these dosages result in serum concentrations of relaxin of about 3, 10, 30 and 75 ng/mL, respectively. In still other embodiments, it is preferable to administer relaxin as about 960 μg/kg of body weight per day. For any dosage level, relaxin may be administered over a period of time sufficient to obtain a therapeutic effect.

In certain embodiments, the methods described above may involve irrigating the wound to speed up healing. Relaxin can be administered in combination with a pharmaceutically acceptable carrier, diluent, or exipient, which may be combined in the form of a lotion, gel, cream, and/or solution. In other embodiments, relaxin is administered in combination with wound penetration enhancers. Relaxin can also be administered in combination with at least one other pharmaceutically active agent, such as an NSAID or an antibiotic. In yet other embodiments, relaxin is administered over a period of time sufficient to obtain a therapeutic effect.

Other aspects of the disclosure provide methods for treating an injury, re-epithelialization of a wound, preventing or reducing scarring during healing of a wound of the skin, wherein pharmaceutically active synthetic human relaxin is administered to a subject. This includes an injectable formulation with doses ranging from about 10 to about 1000 μg/kg of body weight per day, wherein relaxin is administered over a period of time sufficient to obtain a therapeutic effect. In one embodiment, the subject is a human subject. In other embodiments, the injury is a cut, which can be an incision of the epidermis, or a wound, which may be open or closed. Examples of open wounds include, but are not limited to, an incision, a laceration, an abrasion, a puncture wound, a penetration wound, a gunshot wound, and/or a stabbing wound. In yet another embodiment, the formulation is injectable.

In yet other aspects, the disclosure provides methods for preventing or reducing scarring during healing of a wound of the skin, wherein pharmaceutically active synthetic human relaxin is administered to the subject in an amount ranging from about 10 to about 1000 82 g/kg of body weight per day, continuing administration over a period of time sufficient to obtain a therapeutic effect in the subject. In one embodiment, the subject is a human subject. In another embodiment, the wound is an open or a closed wound. In yet another embodiment, the open wound, including but not limited to an incision, a laceration, an abrasion, a puncture wound, a penetration wound, a gunshot wound, and a stabbing wound. In yet another embodiment, scarring includes but is not limited to keloid, hypertrophic, ischemic, and striae. In still another embodiment, pre-existing scar tissue is first removed. In still another embodiment, the formulation is injectable.

The disclosure further encompasses relaxin for use in promoting healing of an injury in a human subject (including diabetic subjects); relaxin for use in promoting healing of a wound in a human subject (including diabetic subjects); relaxin for use in promoting re-epithelialization of a wound in a human subject (including diabetic subjects); relaxin for use in reducing scarring during healing of a wound in a human subject (including diabetic subjects); relaxin for use in preventing scarring during healing of a wound in a human subject (including diabetic subjects); relaxin for use in inhibiting TGF-beta collagen induced fibroblast proliferation and collagen production of a wound of the skin in a human subject (including diabetic subjects); and relaxin for use in improving the appearance of disfiguration in a human subject (including diabetic subjects) as discussed supra.

The present disclosure further provides methods of improving cosmetic appearance of a skin wound comprising: administering a pharmaceutical formulation comprising pharmaceutically active relaxin to a subject with a skin wound in an amount effective to produce a healed wound with an improved cosmetic appearance as compared to a healed wound of an untreated subject. In some embodiments, the healed wound with an improved cosmetic appearance comprises a closer color match with surrounding skin (a smoother texture, reduced distortion of the surrounding skin, greater contour with surrounding skin, and absence of global pathology). In some preferred embodiments, the healed wound with an improved cosmetic appearance further comprises an interwoven arrangement of collagen fibers. In some embodiments, the relaxin is purified, recombinant or synthetic human relaxin. In some preferred embodiments, the relaxin is H1, H2 or H3 human relaxin, while in other embodiments the relaxin is a relaxin agonist. In some preferred embodiments, the relaxin is administered systemically to the subject and/or the relaxin is administered topically to the skin wound. Some embodiments, further comprising irrigating the skin wound. Moreover, in some embodiments, the pharmaceutical formulation further comprises one or both of an antibiotic and a non-steroidal anti-inflammatory drug. In some embodiments, the subject is a human subject with impaired healing capability (e.g., diabetic, aged).

In addition, the present disclosure provides methods for reducing scarring during healing of a skin wound comprising: administering a pharmaceutical formulation comprising pharmaceutically active relaxin to a subject with a skin wound in an amount effective to produce a healed wound with reduced scarring as compared to a healed wound of an untreated subject. In some embodiments, the scarring is selected from the group consisting of a keloid, a hypertrophic scar, and striae. In some preferred embodiments, the methods further comprise debridement or removal of pre-existing scar tissue. In some embodiments, the relaxin is purified, recombinant or synthetic human relaxin. In some embodiments, the relaxin is H1, H2 or H3 human relaxin, while in other embodiments the relaxin is a relaxin agonist. In some preferred embodiments, the relaxin is administered systemically to the subject and/or topically to the skin wound. Some methods further comprise irrigating the skin wound. Moreover, in some embodiments, the pharmaceutical formulation further comprises one or both of an antibiotic and a non-steroidal anti-inflammatory drug. In some embodiments. the subject is a human subject with impaired healing capability (e.g., diabetic, aged).

The present disclosure also provides methods of promoting healing of a wound comprising: administering a pharmaceutical formulation comprising pharmaceutically active relaxin to a subject with a wound in an amount effective to promote healing of the wound. In some embodiments, the wound results from plastic surgery. In some embodiments, the plastic surgery is facial plastic surgery selected from the group consisting of rhytidectomy, blepharoplasty, rhinoplasty, otoplasty, mentoplasty, face lift, fore head lift, brow lift, facial scar revision, facial scar removal, laser surgery, skin resurfacing, wrinkle treatment, plasma skin regeneration, facial fat grafting, skin tightening, tattoo removal and hair replacement. In other embodiments, the plastic surgery is full-body plastic surgery selected from the group consisting of abdominoplasty, breast reduction, breast enhancement, body lift procedures, spider vein treatment, stretch mark treatment, liposuction, excess skin removal surgery, cellulite reduction treatment, body contouring, body resurfacing and body implants. In some embodiments, the relaxin is purified, recombinant or synthetic human relaxin. In some preferred embodiments. the relaxin is H1, H2 or H3 human relaxin, while in other embodiments the relaxin is a relaxin agonist. In some preferred embodiments the relaxin is administered systemically and/or topically. Moreover, in some preferred embodiments the pharmaceutical formulation further comprises one or both of an antibiotic and a non-steroidal anti-inflammatory drug. In some embodiments, the subject is a human subject with impaired healing capability (e.g., diabetic, aged).

### BRIEF DESCRIPTION OF THE DRAWINGS

The present disclosure is best understood when read in conjunction with the accompanying figures, which serve to illustrate the preferred embodiments. It is understood, however, that the disclosure is not limited to the specific embodiments disclosed in the figures.

FIG. 1A depicts the peptide hormone H2 relaxin which is similar in size and shape to insulin. FIG. 1B provides the amino acid sequence of the B chain (SEQ ID NO:1) and the A chain (SEQ ID NO:2 with X representing glutamic acid [E] or glutamine [Q]) of human relaxin 2 (H2).

FIG. 2 depicts the pig wound layout, (A) graphically and (B) photographically from a live pig.

FIG. 3 depicts wound sites on the back of a juvenile pig after six weeks of treatment with relaxin and placebo. Section 3 was irrigated with relaxin during the first week, and subsequently treated with a high dose of topical relaxin formulation during weeks 2-6. Sections 4, 5, and 6 received no irrigation during week 1. Section 4 was treated with placebo. Section 5 was treated with a low dose topical formulation, and Section 6 was treated with high dose topical formulation.

FIG. 4 depicts variations in wound appearance after day six. (A) The top photograph depicts the skin of the control pig. (B) The bottom photograph depicts the skin of the treated pig, wherein systemic relaxin was applied. As can be seen on the figure, the wound treated with relaxin healed faster, better and the appearance of the healed skin appears smoother in comparison to the control pig.

FIG. 5 depicts visual rankings of the cosmetic appearance of the skin: (A) depicts the cosmetic score, and (B) depicts the score for the color match with surrounding skin. Both scores were taken at weeks four and six.

FIG. 6 depicts wound sites on a juvenile pig after six weeks of treatment. The area indicated by the arrows shows areas where (A) no scar was visible when the skin was treated with systemic relaxin, and (B) a red scar was visible when the skin was treated with the placebo. The black marks on the sides of the pictures indicate the extent of the original wound. Scabs visible in the pictures indicate where punch biopsies were taken during the study.

FIG. 7 is a histopathological slide that depicts a wound site after treatment with relaxin.

FIG. 8 shows two histopathological slides of a wound site on a juvenile pig comparing granulation tissue with and without relaxin treatment. Severe scarring was observed in the left slide (no relaxin treatment) and mild scarring was observed in the right slide (relaxin treatment). The slides indicate the wide range in granulation tissue that can be seen after at six weeks following a wound. The slides also illustrate the effect of relaxin in reducing the amount of granulation tissue (right) and the resulting restoration of tissue to a normal appearance.

FIG. 9 shows the scoring of the amount of granulation tissue (left) and inflammation (right) in wound sites after six weeks (blinded assessments). Wounds with systemic relaxin treatment (red color) have significantly reduced granulation tissue or inflammation than wounds without systemic relaxin treatment (blue). This indicates that the wound site resolves better when treated with relaxin via systemic delivery.

FIG. 10 shows slides of collagen repair used to assess wound healing. (A) The top depicts relatively unorganized collagen. (B) The bottom depicts well-healed collagen.

FIG. 11 graphically indicates that the composite wound healing score was significantly improved in wounds treated with systemic relaxin (right) compared with wounds not receiving systemic relaxin (left). The wound score is a three point scale based on the subjective appearance of the collagen in the wound site and a higher number indicates better healing. In determining the score, “1” refers to more fibers arranged in parallel bundles or in one plane, much smaller in size, and more tightly packed. “2” refers to intermediate fiber arrangement and weave. “3” refers to an interwoven arrangement of the collagen fibers similar to the normal pattern, but smaller in size compared to the normal dermis, indicating the best healing.

FIG. 12 shows micrographs that depict the new scar scoring system to classify scars. Normal skin is depicted in (A), mild in (B), severe in (C), and scarring in (D). Collagen bundle orientation is indicated by color: blue, white, and yellow-orange. Mild scars appear woven yet contain thinner collagen. Severe scars have extremely thin collagen bundles, mainly oriented in one plane. The scale is 100 microns, congo red stain, polarized light, 20×.

FIG. 13 depicts the quantitative collagen scoring system. Calculation of the “redness” ratio is accomplished using Photoshop Software. The scar perimeter is traced and the mean number of red pixels is calculated. An adjacent, normal area is traced and the mean number of red pixels is calculated.

FIG. 14 indicates the results of the objective collagen scoring system applied to this study. Wounds that received systemic relaxin (right) had significantly better collagen organization than wounds that did not receive systemic relaxin (left).

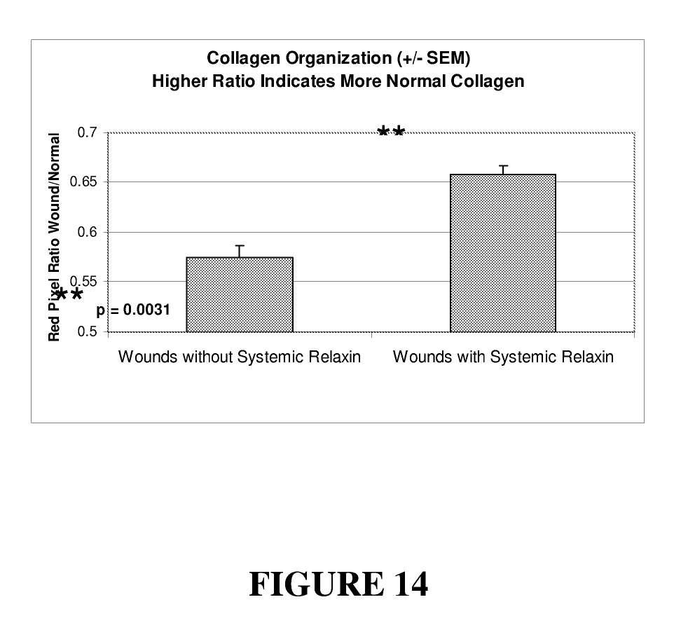

FIG. 15 depicts the concentration of H2 relaxin (H2 RLX) detected in treated dog gingiva measured at 0.5 cm, 1.0 cm and 2.0 cm from the site of injection, at 1 hour, 2 hours and 4 hours after injection. Assessment of the treated dog gingiva revealed that relaxin (RLX) was present at all three time points, but a steady decrease in concentration was observed at 1, 2 and 4 hours. Specifically, 90 nM, 78 nM and 21 nM of H2 RLX were detected, respectively. RLX was identified to have limited migration through the gingiva. At the 1 hour time point RLX was detected at 0.5 cm from the site of injection but was not detected at 1.0 and 2.0 cm from the site of injection. At the remaining time points RLX was only detected at the site of injection. Thus, this figure clearly shows that relaxin stays close to the injection site.

### DETAILED DESCRIPTION

### General Overview

The present disclosure relates to methods of promoting healing of an injury or a wound. The disclosure presents methods for treating an injury, promoting re-epithelialization of a wound, as well as reducing and preventing scarring during the healing process. The disclosure further provides methods for inhibiting TGF-beta collagen induced fibroblast proliferation and collagen production, and improving the appearance of skin disfiguration. In certain embodiments, the methods of the present disclosure are particularly suitable for applications in the wound acceleration and strengthening arena, for example, in patients who have poor healing after surgery or injury such as the elderly, and patients with co-morbidities such as diabetic and immuno-compromised patients. In other embodiments, the methods of the present disclosure find applicability in cosmetic surgery.

The disclosure presents a significant advancement in the field of wound healing and scar prevention. The addition of relaxin to wounds in clinical and pre-clinical settings accelerates the healing process, particularly the rate of re-epithelialization. Treatment with relaxin is also therapeutically effective in the reduction and prevention of scarring. The binding of relaxin to specific G-protein coupled relaxin receptors, such as LGR7 and LGR8, is believed to result in better wound healing and scar prevention as relaxin is capable of modulation these receptors.

Relaxin also promotes new blood vessel growth (angiogenesis) in wound sites via the induction of angiogenic cytokines, such as vascular endothelial growth factor (VEGF) and fibroblast growth factors (FGF). Stimulating the release of VEGF promotes mitotic and/or migratory activity of endothelial cells. Also critical to tissue repair is the establishment of the extracellular scaffold to support cell migration and/or proliferation. Stimulating the release of FGFs from any of a number of cell types promotes proliferation and migration of fibroblasts, which are involved in the production of extracellular matrix (ECM) materials such as collagen. Surprisingly, the inventor found that relaxin leads to an overall reduction in blood vessels in the wound site after about six weeks of treatment, i.e., there is significantly less redness seen in the wound site as a result of relaxin treatment. This is a novel finding which is intriguing since relaxin first promotes new blood vessel growth in a fresh wound site and then reduces these new blood vessels after about six weeks of treatment, leading to a finer, smoother and more normal looking skin.

### Definitions

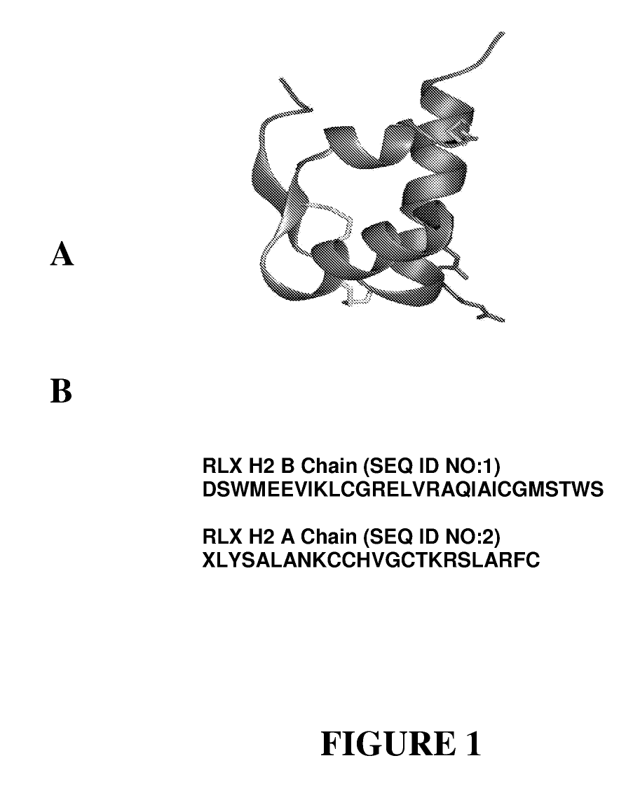

The term “relaxin” refers to a peptide hormone which is well known in the art (see FIG. 1). The term “relaxin”, as used herein, encompasses human relaxin, including intact full length human relaxin or a portion of the relaxin molecule that retains biological activity. The term “relaxin” further contemplates synthetic human relaxin and recombinant human relaxin, including synthetic H2 human relaxin and recombinant H2 human relaxin. The term further encompasses active agents with relaxin-like activity, such as relaxin analogs and portions thereof that retain biological activity, and agents that competitively displace bound relaxin from a relaxin receptor such as an LGR7 or an LGR8 receptor. In addition, the nucleic acid sequence of human relaxin as used herein must not be 100% identical to nucleic acid sequence of human relaxin H2 but may be at least about 40%, 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%. 97%, 98% or 99% identical to the nucleic acid sequence of human relaxin H2. Relaxin, as used herein, can be made by any method known to those skilled in the art. Examples of such methods are illustrated, for example, in U.S. Pat. No. 5,759,807, as well as in Büllesbach et al., J Biol Chem, 266: 10754-10761, 1991. Examples of relaxin molecules and analogs are illustrated, for example. in U.S. Pat. 5,166,191.

The term “wound” generally refers to both open and closed wounds, as defined below. A wound can be further classified as an acute or chronic wound. An acute wound is one that does not have an underlying healing defect, and usually occurs secondarily to surgery or trauma in a healthy individual, healing quickly and completely. In contrast, a chronic wound is one that has a loss in tissue integrity, produced by insult or injury that is of extended duration or frequent recurrence. As used herein, the term “skin wound” refers to a break in the skin.

The term “open wound” is usually classified according to the object that caused the wound. This includes incisions, lacerations, abrasions, puncture wounds, penetration wounds, gunshot wounds and the like. Incisions or incised wounds may be caused by a clean, sharp-edged object such as a knife, a razor. or a glass splinter. Incisions involving only the epidermis can be classified as cuts. Lacerations are irregular wounds caused by a blunt impact to soft tissue that lies over hard tissue (such as laceration of the skin covering the skull) or tearing of skin and other tissues (such as caused by childbirth). Lacerations may show bridging, as connective tissue or blood vessels are flattened against the underlying hard surface. Abrasions (grazes) are superficial wounds in which the topmost layer of the skin (the epidermis) is scraped off, and are often caused by a sliding fall onto a rough surface. Puncture wounds may be caused by an object puncturing the skin, such as a nail or needle. Penetration wounds may be caused by an object such as a knife entering the body. Gunshot wounds are caused by a bullet or similar projectile driving into or through the body. As such, there may be two wounds, one at the site of entry and one at the site of exit, which is generally known as a through-and-through.

The term “closed wound” refers to contusions, more commonly known as bruises, caused by blunt force trauma that damages tissue under the skin; hematomas, also called blood tumors, caused by damage to a blood vessel that in turn causes blood to collect under the skin; and crushing injuries, which may be caused by a great or extreme amount of force applied over a long period of time.

The term “scar” refers to an abnormal morphological structure resulting from a previous injury or wound (e.g., an incision, excision or trauma). Scars are composed of a connective tissue which is predominately a matrix of collagen types 1 and 3 and fibronectin. A scar may consist of collagen fibers in an abnormal organization (as seen in normal scars of the skin) or may be an abnormal accumulation of connective tissue (as seen in scars of the central nervous system or pathological scarring of the skin). The types of scars include, but are not limited to, atrophic, hypertrophic and keloidal scars, as well as scar contractures. Atrophic scars are flat and depressed below the surrounding skin as a valley or hole. Hypertrophic scars are elevated scars that remain within the boundaries of the original lesion, and often contain excessive collagen arranged in an abnormal pattern. Keloidal scars are elevated scars that spread beyond the margins of the original wound and invade the surrounding normal skin in a way that is site specific, and often contain whorls of collagen arranged in an abnormal fashion. Scar contractures are scars that cross joints or skin creases at right angles, and are prone to developing shortening or contracture. Scar contractures occur when the scar is not fully matured, often tend to be hypertrophic, and are typically disabling and dysfunctional.

“Administering” refers to giving or applying to a subject a pharmaceutical remedy or formulation via a specific route, including but not limited to, topically, intravenously, systemically, subcutaneously, intramuscularly, sublingually, and via inhalation or injection, irrigation or an osmotic pump.

### Relaxin

Relaxin is a peptide hormone that is similar in size and shape to insulin (see FIG. 1). More specifically, relaxin is an endocrine and autocrine/paracrine hormone which belongs to the insulin gene superfamily. The active form of the encoded protein consists of an A chain and a B chain, held together by disulphide bonds, two inter-chains and one intra-chain. Thus, the structure closely resembles insulin in the disposition of disulphide bonds. In humans, there are three non-allelic relaxin genes, relaxin-1 (RLN-1 or H1), relaxin-2 (RLN-2 or H2) and relaxin-3 (RLN-3 or H3). H1 and H2 share high sequence homology. There are two alternatively spliced transcript variants encoding different isoforms described for this gene. Expression of H1 in humans is uncertain. H2 is expressed in reproductive organs while H3 is found primarily in the brain. The evolution of the relaxin peptide family in its receptors is generally well known in the art (Wilkinson et al., BMC Evolutionary Biology, 5:1-17, 2005; and Wilkinson and Bathgate, Chapter 1, Relaxin and Related Peptides, Landes Bioscience and Springer Science+Business Media, 2007).

Relaxin activates two specific relaxin receptors, i.e., LGR7 (RXFP1) and LGR8 (RXFP2). LGR7 and LGR8 are leucine-rich repeat-containing, G protein-coupled receptors (LGRs) which represent a unique subgroup of G protein-coupled receptors. They contain a heptahelical transmembrane domain and a large glycosylated ectodomain, distantly related to the receptors for the glycoproteohormones, such as the LH-receptor or FSH-receptor. These relaxin receptors are found in the heart, smooth muscle, connective tissue, and central and autonomous nervous system. Potent relaxins such as H1, H2, porcine and whale relaxin possess a certain sequence in common, i.e., the Arg-Glu-Leu-Val-Arg-X-X-Ile sequence or binding cassette. Relaxins that deviate from his sequence homology such as rat, shark, dog and horse relaxins show a reduction in bioactivity through the LGR7 and LGR8 receptors (Bathgate et al., Ann NY Acad Sci, 1041: 61-76, 2005).

Relaxin is found in both, women and men (Tregear et al., Relaxin 2000, Proceedings of the Third International Conference on Relaxin & Related Peptides, 22-27 Oct. 2000, Broome, Australia). In women, relaxin is produced by the corpus luteum of the ovary, the breast and, during pregnancy, also by the placenta, chorion, and decidua. In men, relaxin is produced in the testes. In humans, relaxin plays a role in pregnancy, in enhancing sperm motility, regulating blood pressure, controlling heart rate and releasing oxytocin and vasopressin. In animals, relaxin also affects collagen metabolism, inhibiting collagen synthesis and enhancing its breakdown by increasing matrix metalloproteinases. Relaxin also enhances angiogenesis and is a renal vasodilator.

Relaxin has the general properties of a growth factor and is capable of altering the nature of connective tissue and influencing smooth muscle contraction. H2 is known to be primarily expressed in reproductive tissue (see U.S. Pat. No. 5,023,321). However, the inventor has discovered that H2 plays a major role in improving wound healing and reducing scar formation.

### Relaxin Agonists

In some embodiments, the present disclosure provides methods of treating dyspnea associated with acute heart failure in normotensive or hypertensive patients comprising administration of a relaxin agonist. In some methods, the relaxin agonist activates one or more relaxin-related G-protein coupled receptors (GPCR) selected from but not limited to RXFP1, RXFP2, RXFP3, RXFP4, FSHR (LGRI), LHCGR (LGR2), TSHR (LGR3), LGR4, LGR5, LGR6 LGR7 (RXFP1) and LGR8 (RXFP2). In some embodiments, the relaxin agonist comprises the amino acid sequence of Formula I of WO 2009/007848 of Compugen (herein incorporated by reference for the teaching of relaxin agonist sequences).

Formula I peptides are preferably from 7 to 100 amino acids in length and comprise the amino acid sequence: X1-X2-X3-X4-X5-X6-X7-X8-X9-X10-X11-X12-X13-X14-X15-X16-X17-X18-X19-X20-X21-X22-X23-X24-X25-X26-X27-X28-X29-X30-X31-X32-X33; wherein X1 is absent or G or a small naturally or non-naturally occurring amino acid: X2 is absent or Q or a polar naturally or non-naturally occurring amino acid; X3 is absent or K or a basic naturally or non-naturally occurring amino acid; X4 is absent or G or a small naturally or non-naturally occurring amino acid; X5 is absent or Q or S a polar naturally or non-naturally occurring amino acid; X6 is absent or V or A or P or M or a hydrophobic naturally or non-naturally occurring amino acid; X7 is absent or G or a small naturally or non-naturally occurring amino acid; X8 is absent or P or L or A naturally or non-naturally occurring amino acid; X9 is absent or P or Q naturally or non-naturally occurring amino acid; X10 is absent or G or a small naturally or non-naturally occurring amino acid; X11 is absent or A or H or E or D or a hydrophobic or a small or an acidic naturally or non-naturally occurring amino acid; X12 is absent or A or P or Q or S or R or H or a hydrophobic or a small naturally or non-naturally occurring amino acid; X13 is absent or C or V or a hydrophobic naturally or non-naturally occurring amino acid; X14 is absent or R or K or Q or P or a basic or a polar naturally or non-naturally naturally occurring amino acid; X15 is absent or R or Q or S or a basic or a polar naturally or non-naturally occurring amino acid; X16 is absent or A or L or H or Q or a hydrophobic or a small naturally or non-naturally occurring amino acid; X17 is absent or Y or a hydrophobic or an aromatic naturally or non-naturally occurring amino acid; X18 is absent or A or a hydrophobic or small naturally or non-naturally occurring amino acid; X19 is absent or A or a hydrophobic small naturally or non-naturally occurring amino acid; X20 is absent or F or a hydrophobic or an aromatic naturally or non-naturally occurring amino acid; X21 is absent or S or T or a polar naturally or non-naturally occurring amino acid; X22 is absent or V or a hydrophobic naturally or non-naturally occurring amino acid; X23 is absent or G or hydrophobic or small non-naturally occurring amino acid or replaced by an amide; X24 is absent or R or a basic naturally or non-naturally occurring amino acid; X25 is absent or R or a basic naturally or non-naturally occurring amino acid; X26 is A or a hydrophobic or small naturally or non-naturally occurring amino acid; X27 is Y or a hydrophobic or an aromatic naturally or non-naturally occurring amino acid; X28 is A or a hydrophobic or small naturally or non-naturally occurring amino acid; X29 is A or a hydrophobic or small naturally or non-naturally occurring amino acid; X30 is F or a hydrophobic naturally or non-naturally occurring amino acid; X31 is S or T or a polar naturally or non- naturally occurring amino acid; X32 is V or a hydrophobic naturally or non-naturally occurring amino acid; X33 is absent or G or hydrophobic or small naturally or non-naturally occurring amino acid or replaced by an amide; or a pharmaceutically acceptable salt thereof (SEQ ID NO:4). In some preferred embodiments, the relaxin agonist comprises the sequence of peptide P59C13V (free acid) GQKGQVGPPGAA VRRA Y AAFSV (SEQ ID NO:5). In another preferred embodiment, the relaxin agonist comprises the sequence of peptide P74C13V (free acid) GQKGQVGPPGAA VRRA Y AAFS VGRRA Y AAFS V (SEQ DD NO: 6). Further derivatives of the human complement C1Q tumor necrosis factor-related protein 8 (CTRP8 or C1QT8) such as peptide P59-G (free acid Gly) GQKGQVGPPGAACRRA Y AAFSVG (SEQ ID NO:7) are also contemplated to be suitable for use in the methods of the present disclosure. The amino acid sequence of C1QT8 is set forth as SEQ ID NO:8 MAAPALLLLALLLPVGAWPGLPRRPCVHCCRPAWPPGPYARVSDRDLWRGDLWRGLP RVRPTIDIEILKGEKGEAGVRGRAGRSGKEGPPGARGLQGRRGQKGQVGPPGAACRRA YAAFSVGRRAYAAFSVGRREGLHSSDHFQAVPFDTELVNLDGAFDLAAGRFLCTVPGV YFLSLNVHTWNYKETYLHIMLNRRPAAVLYAQPSERSVMQAQSLMLLLAAGDAVWVR MF QRDRDNAIYGEHGDLYITFSGHLVKP AAEL.

The present disclosure also encompasses homologues of these polypeptides, such homologues can be at least 50%, at least 55%, at least 60%, at least 65%, at least 70%, at least 75%, at least 80%, at least 85%, at least 85%, at least 90%, at least 95% or more say 100% identical to the amino acid sequence of an exemplary relaxin agonist (e.g., SEQ ID NO:5 or SEQ ID NO:6), as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and (initialization and extension). Optionally and preferably, nucleic acid sequence identity/homology is determined with BlastN software of the National Center of Biotechnology Information (NCBI) using default parameters, which preferably include using the DUST filter program, and also preferably include having an E value of 10, filtering low complexity sequences and a word size of 11. Finally the present disclosure also encompasses fragments of the above described polypeptides and polypeptides having mutations, such as deletions, insertions or substitutions of one or more amino acids, either naturally occurring or artificially induced, either randomly or in a targeted fashion.

### Relaxin Treatment Promotes Wound Healing

When the skin is injured, for example, due to a cut, a wet wound is generated, and the body's natural process to regenerate dermal and epidermal tissue to repair the damage consists of a set of complex biochemical events. Relaxin enhances and speeds up this process. In the early stages of wound healing, once the bacteria and debris are removed from the wound site, new blood vessels grow from endothelial cells through angiogenesis. Furthermore, fibroblasts grow and form a new extracellular matrix (ECM) by excreting collagen and fibronectin. The disclosure provides methods to speed up the natural wound healing process and increase the strength of a wound site by increasing granulation tissue formation, eventually reducing the number of blood vessels that result from angiogenesis, increasing collagen deposition, aiding in the formation of a better organized collagen matrix, and promoting the re-epithelialization of a wound. In some instances, when wounds do not heal properly (e.g., in diabetics, in individuals with skin disorder), relaxin treatment not only enhances wound healing but makes it possible.

More specifically, the inventor has discovered that relaxin is advantageous in its ability to promote wound healing in a two-step process. First, relaxin activates fibroblasts and promotes angiogenesis for good healing to quickly close a wound. Second, relaxin has anti-fibrotic and anti-angiogenic effects to achieve smoother healing, thereby reducing scar formation. The fact that relaxin has anti-angiogenic effects is a novel finding that is contrary to what is generally believed, namely, that relaxin only promotes growth of blood vessels and thereby enhances angiogenesis.

Fibroblasts play a critical role in maintaining the structural integrity of connective tissues by continuously secreting precursors of the ECM. Under normal conditions, fibroblasts are in a resting state. However, when fibroblasts are activated in a wet wound, they secrete more collagen. Relaxin participates in the activation of fibroblasts, which leads to increased secretion of collagen. Fibroblasts also secrete matrix metalloproteinases (MMPs), which help break down collagen. Relaxin can also inhibit TGF-beta collagen induced fibroblast proliferation and collagen production. Thus, this combination of increased secretion of collagen and increased turnover of collagen leads to a more organized and stronger collagen matrix that promotes wound healing and reduces scar formation.

Angiogenesis occurs very early during the wound healing process. While macrophages and lymphocytes are recruited to the wound site, new blood vessels form to provide the wound with enough blood supply. Specifically, macrophages release VEGF which stimulates formation of new blood vessels. When relaxin is used for treatment, the amount of VEGF is increased leading to more blood vessels and thus, more blood supply to the wound.

The entire process of recruiting the immune system, forming new blood vessels (angiogenesis) and activating fibroblasts is collectively considered the granulation process—i.e., a process by which granulation tissue is generated. At some point, this granulation must be resolved in order to ultimately achieve the desirable basket weave pattern that is present in normal, healthy, smooth skin. This entails slowing down the fibroblast since no additional collagen is needed, and slowing down the immune system since inflammation needs to go down so the red marks in the skin can be resolved. In addition, angiogenesis needs to be reversed, i.e., the blood vessels and capillaries that were needed to heal the wound in the first place now need to disappear to prevent unsightly permanent red marks scars. Surprisingly, relaxin not only enhances and speeds up the resolving of granulation, but it also fosters the early disappearance of the new blood vessels, leaving the skin smooth and free of red marks. Notably, relaxin treatment dramatically reduces the formation of scars and enhances the healthy looking basket weave pattern of the skin.

Furthermore, relaxin can promote wound healing in bruises and ulcers. Such wounds are preferably treated systemically because topical relaxin treatment does not get into closed wounds as easily as into open or wet wounds. However, open and wet wounds are preferably treated topically, via irrigation and/or direct injection (as illustrated in the Pig Skin Study of Example 1).

### Relaxin Treatment Prevents and Reduces Scar Formation

Scars include fibrous tissue and replace normal skin after injury or a wound. Scarring is a natural part of the wound healing process, and scars arise after almost every dermal injury. Each year in the developed world, 100 million patients acquire scars, some of which cause considerable health as well as psychological problems, as a result of 55 million elective operations and 25 million operations after trauma. There are an estimated 11 million keloid scars, and 4 million burn scars, 70% of which occur in children. People with abnormal skin scarring may face physical, aesthetic, psychological, and social consequences that may be associated with substantial emotional and financial costs. While scars are often considered trivial, they can be disfiguring and aesthetically unpleasant and cause severe itching, tenderness, pain, sleep disturbance, anxiety, depression, and disruption of daily activities. Other psychological consequences include development of post-traumatic stress reactions, loss of self esteem, and stigmatization, leading to diminished quality of life. Physical deformity as a result of skin scars can be disabling. In spite of media suggestions to the contrary, scars cannot yet be easily removed because of the complex and unpredictable nature of scar tissue (Bayat et al., Clinical Review—BMJ, 326: 88, 2003). Relaxin provides a method for treating such scars not only by speeding up the wound healing process, but also by reducing the scar formation before it begins.

Scars commonly form as a result of facial plastic surgery, which includes, but is not limited to, rhytidectomy, blepharoplasty, rhinoplasty, otoplasty, mentoplasty, face lift, fore head lift, brow lift, facial scar revision, facial scar removal, laser surgery, skin resurfacing, wrinkle treatment, plasma skin regeneration, facial fat grafting, skin tightening, tattoo removal and hair replacement. In addition, facial plastic surgery can often result in swelling, bruising, and scarring. As such, relaxin is particularly applicable in plastic and reconstructive surgery. Swelling is the face's natural reaction to an injury, and will subside as the face begins to heal itself, which may occur just a few days after surgery or mat take up to several weeks or longer. Bruising is also natural following a facial surgery procedure as the face reacts to the changes, and is usually most pronounced in the first few days of recovery after surgery. While most bruising will vanish in a couple of weeks. full healing may take months or longer, depending on the individual. Scarring is yet another unpleasant side effect of facial plastic surgery, as scars will usually remain pink for several months before becoming less noticeable. Thus, this disclosure is advantageous to patients who undergo facial plastic surgery, particularly to aid with scarring and bruising, by speeding up wound healing and reducing scar formation.

Scars also commonly form as a result of full-body plastic surgery, which includes, but is not limited to, abdominoplasty, breast reduction, breast enhancement, body lift procedures, spider vein treatment, stretch mark treatment, liposuction, excess skin removal surgery, cellulite reduction treatment, body contouring, body resurfacing and body implants. Full-body plastic surgery often also results in swelling, bruising, and scarring. Swelling is the body's reaction to an injury, and usually subsides as the body begins to heal itself, which may occur just a few days after surgery or up to several weeks or longer. Bruising normally occurs as a result of full-body surgery procedures as the body reacts to the changes. Bruising is usually most pronounced in the first few days of recovery after surgery but may last longer. While most bruising may vanish in a couple of weeks, full healing may take months or even years. Scarring is also an unpleasant side effect of full body surgery, as scars will usually remain pink for several months before becoming less noticeable. Thus, this disclosure is also beneficial to patients who undergo full-body plastic surgery, particularly to aid with scarring and bruising, by speeding up wound healing and reducing scar formation.

Scarring and redness are also common side effects of tattoo removal. Other side effects include blistering, infection, and loss of skin color. Thus, this disclosure provides a method to minimize some of the side effects of tattoo removal, particularly by reducing the skin's redness (i.e., relaxin's anti-angiogenesis effect as discussed, supra) and by reducing any scars that may result from the tattoo removal procedure.

### Relaxin Compositions and Formulations

Relaxin and relaxin analogs are formulated as pharmaceuticals to be used in the methods of the disclosure. Any composition or compound that can stimulate a biological response associated with the binding of biologically or pharmaceutically active relaxin (e.g., synthetic relaxin, recombinant relaxin) or a relaxin agonist (e.g., relaxin analog or relaxin-like modulator) to relaxin receptors can be used as a pharmaceutical in the disclosure. General details on techniques for formulation and administration are well described in the scientific literature (see Remington's Pharmaceutical Sciences, Maack Publishing Co, Easton Pa.). Pharmaceutical formulations containing pharmaceutically active relaxin can be prepared according to any method known in the art for the manufacture of pharmaceuticals. The formulations containing pharmaceutically active relaxin or relaxin agonists used in the methods of the disclosure can be formulated for administration in any conventionally acceptable way including, but not limited to, topically, intravenously. systemically, subcutaneously. intramuscularly. sublingually, and via inhalation or injection, irrigation or an osmotic pump. Illustrative examples are set forth below.

In one preferred embodiment, relaxin may be applied either as an irrigation fluid to an open wound, as a topical application to a wound that has covered, and/or systemically during any stage of wound repair. It is considered that a formulation could be injected near the wound site or scar, or may be a cream that would be rubbed in to a wound site or scar to lengthen residence time and penetration. Treatment via irrigation involves, for example, slowly dripping 0.5 ml of a 1.0 mg/ml relaxin solution in sodium acetate onto the wound site. In other embodiments, the ranges for dripping relaxin solutions onto wounds may range from about 0.5 ml to about 5.0 ml or higher of about 1.0 to about 5.0 mg/ml relaxin solutions onto a wound site. Wounds are irrigated, for example, once daily for seven days following the lesion. In another embodiment, wounds are irrigated, for example, twice or more often per day for about seven days following the lesion. In yet other embodiments, wounds are irrigated weekly or monthly for a longer or shorter duration following the lesion. Dosages for purposes of relaxin administration have to be adjusted according to the severity of the lesion and condition of the patient.

In yet another embodiment, relaxin may be applied topically, for example, with a delivery of 0.5 ml of either 0.5 or 2.5 mg/ml relaxin in a formulation consisting of 76.5% 20 mM sodium acetate buffer, 0.17% methylparaben, 0.03% propylparaben, 5% propylene glycol, 5% ethanol, 1% HED 250HX and 12.285% relaxin in sodium acetate buffer. The wounds are treated, for example, twice daily for 2 weeks followed by once daily treatment for 3 weeks. When the drugs are delivered topically, the formulation contains propylene glycol and ethanol. Furthermore, the inventor has observed that the efficacy of relaxin is improved when the formulation includes penetration enhancers, which may attach to the wet wound. Penetration enhancers include, but or not limited to physical (e.g., microneedle arrays), chemical (e.g., ethanol, glyceryl monoethyl ether, monoglycerides, isopropylmyristate etc.) or combinations of physical and chemical enhancements. For example, the inventor has noticed that the addition of mucoadhesive penetration enhancers, such as chitosan, increased the penetration of relaxin through gingival tissues (see Squier et al; Mucoadhesive vehicles for the delivery of relaxin across oral mucosa, The International Association for Dental Research, 28 June-1 Jul., 2006, Brisbane, Australia).

In yet another preferred embodiment, following a systemic approach, relaxin is delivered directly into wound by osmotic infusion pump at 5.3 ug/kg/hr to achieve about 20 ng/ml systemic concentrations.

In yet another preferred embodiment, relaxin can be delivered to the wound site by intravenous injection, wherein the formulations contain pharmaceutically active relaxin or a relaxin agonist can be in the form of a sterile injectable preparation, such as a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a nontoxic parenterally-acceptable diluent or solvent. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic monoglycerides or diglycerides. In addition, fatty acids such as oleic acid can likewise be used in the preparation of injectables.

Aqueous suspensions of the disclosure contain relaxin in admixture with excipients suitable for the manufacture of aqueous suspensions. Such excipients include a suspending agent, such as sodium carboxymethylcellulose, methylcellulose, hydroxypropylnethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending relaxin in a vegetable oil, such as arachis oil, olive oil, sesame oil or coconut oil, or in a mineral oil such as liquid paraffin. The oil suspensions can contain a thickening agent, such as beeswax, hard paraffin or cetyl alcohol. Sweetening agents can be added to provide a palatable oral preparation. These formulations can be preserved by the addition of an antioxidant such as ascorbic acid.

Dispersible powders and granules of the disclosure suitable for preparation of an aqueous suspension by the addition of water can be formulated from relaxin in admixture with a dispersing, suspending and/or wetting agent, and one or more preservatives. Suitable dispersing or wetting agents and suspending agents are exemplified by those disclosed above. Additional excipients. for example sweetening, flavoring and coloring agents, can also be present.

The pharmaceutical formulations of the disclosure can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin. esters or partial esters derived from fatty acids and hexitol anhydrides, such as sorbitan mono-oleate. and condensation products of these partial esters with ethylene oxide, such as polyoxyethylene sorbitan mono-oleate. The emulsion can also contain sweetening and flavoring agents. Syrups and elixirs can be formulated with sweetening agents, such as glycerol, sorbitol or sucrose. Such formulations can also contain a demulcent, a preservative, a flavoring or a coloring agent.

### Administration and Dosing Regimen of Relaxin Formulations

The formulations containing pharmaceutically active relaxin used in the methods of the disclosure can be administered in any conventionally acceptable way including, but not limited to. intravenously, subcutaneously, intramuscularly, sublingually, topically, orally and via inhalation. Administration will vary with the pharmacokinetics and other properties of the drugs and the patients' condition of health. General guidelines are presented below.

The methods of the disclosure promote healing of wounds and injury, and reduce scar formation. The amount of relaxin alone or in combination with another pharmaceutically active agent (such as an NSAID or antibiotic) that is adequate to accomplish these effects is considered the therapeutically effective dose (e.g., the pharmaceutically acceptable amount to promote healing).

The state of the art allows the clinician to determine the dosage regimen of relaxin for each individual animal or human subject. As an illustrative example, the guidelines provided below for relaxin can be used as guidance to determine the dosage regimen, i.e., dose schedule and dosage levels, of formulations containing pharmaceutically active relaxin administered when practicing the methods of the disclosure. Particularly, such subjects receive pharmaceutically active H2 human relaxin (e.g., synthetic, recombinant) in an amount in a range of about 10 to 1000 μg/kg of subject body weight per day. In one embodiment, the dosages of relaxin are 10, 30, 100 and 250 μg/kg/day. In another embodiment, these dosages result in serum concentrations of relaxin of about 3, 10, 30 and 75 ng/mL, respectively. In another embodiment, the administration of relaxin is continued as to maintain a serum concentration of relaxin of from about 0.5 to about 500 ng/mL, more preferably from about 0.5 to about 300 ng/mL, and most preferably from about 3 to about 75 ng/mL. A possible range is about 1 to about 50 ng/mL, wherein a preferred serum concentration is 20 ng/mL.

As a general guideline, it is expected that the daily dose of pharmaceutically active 112 human relaxin (e.g., synthetic, recombinant) is typically in an amount in a range of about 10 to 1000 μg/kg of subject body weight per day, and most preferably at about 960 μg/kg of subject body weight per day. Depending on the subject, relaxin administration is maintained for as specific period of time or for as long as needed to achieve stability in the subject.

### EXPERIMENTAL

The following specific examples are intended to illustrate the disclosure and should not be construed as limiting the scope of the claims.

### EXAMPLE 1

### Porcine Skin Wound Study

Overview of the Animal Study. In selecting the appropriate animal for this study, a pig was selected because of the anatomical similarities between pig skin and human skin. In fact, pig wound healing has been used in numerous studies to simulate human skin (Sullivan et al., Wound Repair Regen. 9: 66-76, 2001). The results of this study suggest that in pig skin, relaxin reduces granulation tissue, as well as chronic inflammation, thereby resulting in an overall faster healing process and smoother appearance of skin.

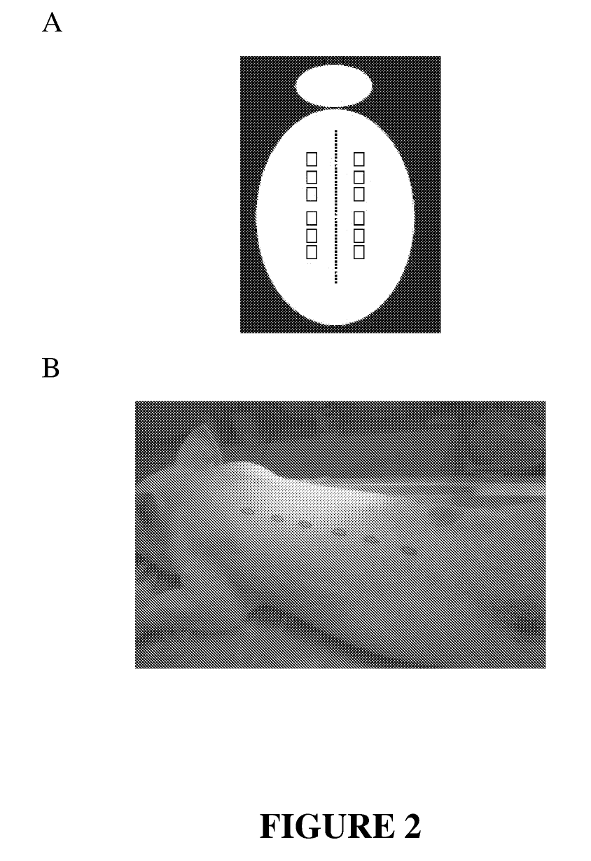

Design of Study. In this study, juvenile pigs were oriented with twelve wound sites per animal—20×6 mm incisional wounds on the back—over the course of a six week study (see FIG. 2). Systemic relaxin was delivered via mini-pumps to achieve 20 ng/mL serum concentrations. Topical formulations were prepared by Dow Pharma, and consisted of propylene glycol and ethanol, which contributed to good long term stability of the therapeutic agent. Dosages consisting of placebo, 0.5 mg/mL (low dose), and 2.5 mg/mL (high dose) were prepared. The goals of the study were to assess the safety of topical relaxin on wound healing and to determine the efficacy of relaxin as a treatment for wound healing and/or scar formation. During the first week, irrigation was performed once daily. During weeks 2-3, topical formulation was applied twice daily, and during weeks 3-6, topical formulation was applied once daily.

Design of Drug. The study drug was relaxin (produced by recombinant technology). Recombinant relaxin is identical to the native human hormone H2 relaxin, produced using a recombinant single chain process. The active test article may be aseptically diluted to the desired concentration with the acetate diluent (20 mM sodium acetate, pH 5.0).

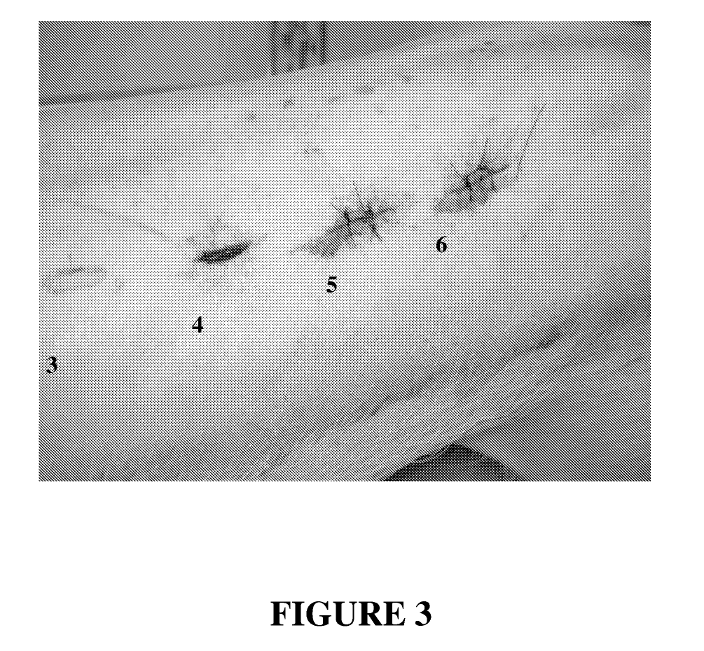

Study Procedures. Referring to FIG. 3 which depicts the back of a pig with incisions according to the experimental design described herein at the completion of six weeks, the wound in Section 3 was irrigated with relaxin construct during the first week, and subsequently treated with high dose topical relaxin formulation during weeks 2-6 as described above. Sections 4, 5, and 6 received no irrigation during week 1. Section 4 was treated with placebo. Section 5 was treated with low dose topical formulation, and Section 6 was treated with high dose topical formulation.

Comparison of section 4 with each of sections 5 and 6 clearly demonstrate the improved wound healing from topical-only treatment with relaxin compared to placebo. Even more dramatic is comparison to section 3, which received irrigation treatment with relaxin in addition to the topical treatment. The experiment shows the remarkable power of relaxin to promote wound healing and to reduce and/or prevent scarring compared to placebo. The data also show that irrigation with relaxin construct is particularly efficacious.

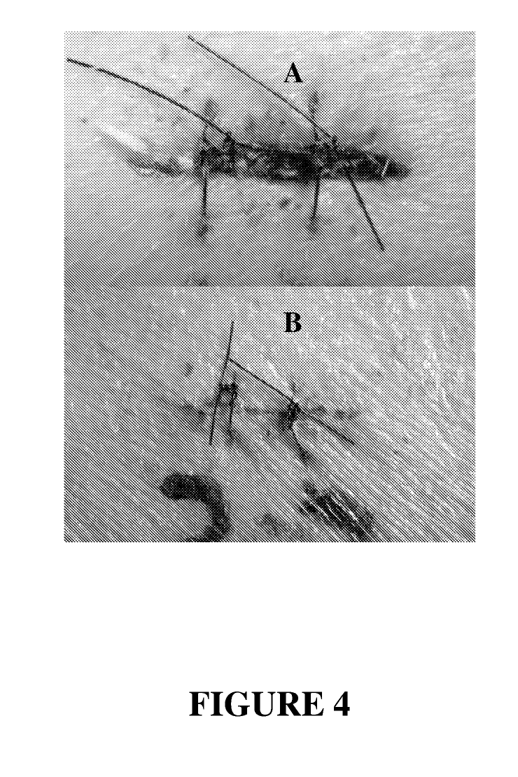

Cosmetic appearance of skin. Visual rankings of wound pictures were conducted by neutral observers who evaluated scabs during early wound healing (days 5-7) and scars at the end of the study (see FIG. 4). Pictures were shown to non-dermatologists for assessment. Observers were blinded to treatments and asked to assess the pictures by their own criteria, choosing and ranking the best three groups among six groups (each with eight photos), wherein three groups received relaxin and three did not. The results of this visual wound assessment determined that 89% of the time a relaxin treatment group was selected in the top three. Only 11% of the time, a group not treated with relaxin was selected in the top three. Neutral observers selected wounds treated with relaxin as appearing “better.” These data strongly suggested that relaxin will help in the early closure of wounds.

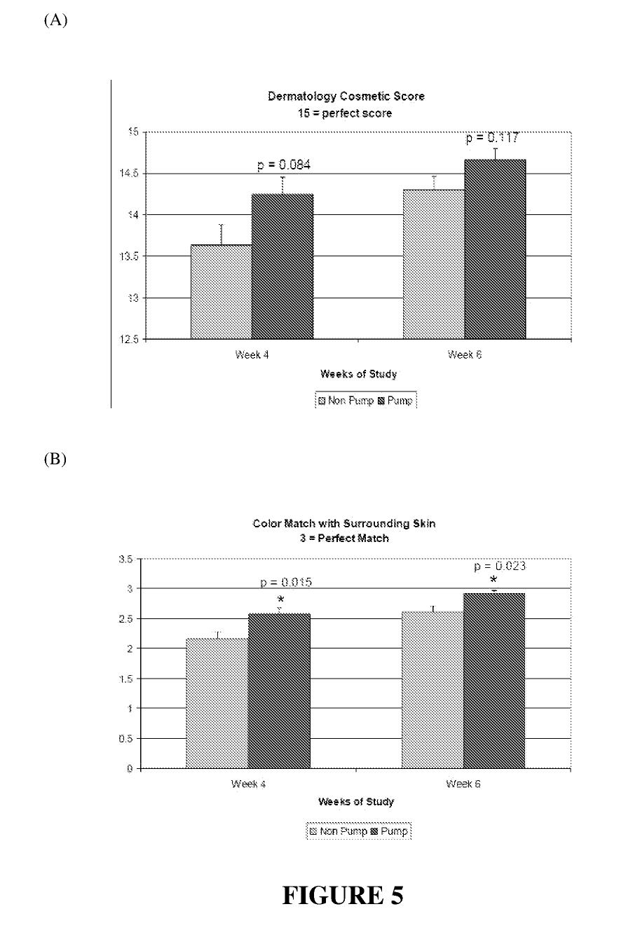

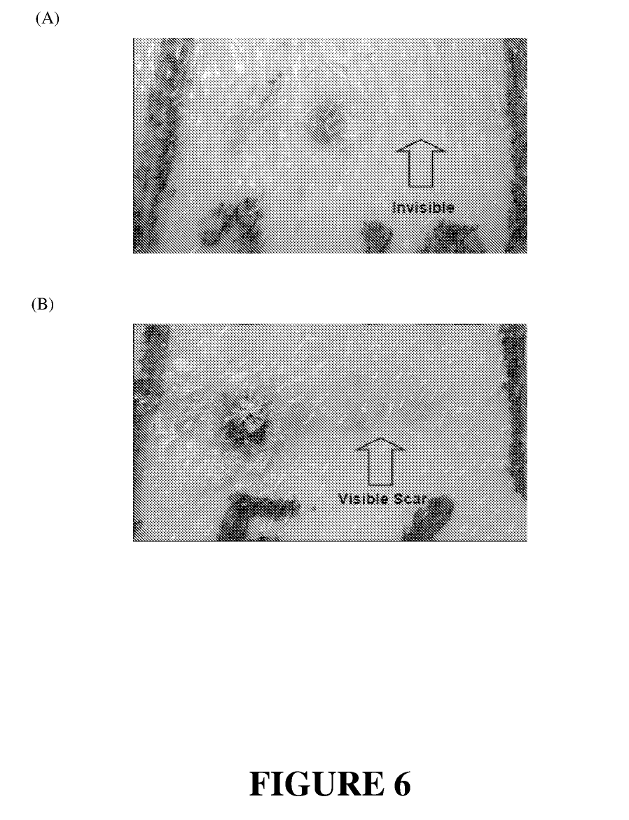

A cosmetic score was also determined by a professional dermatologist at weeks 2, 4, and 6. The cosmetic score has five components: (1) color—matching with surrounding skin; (2) texture—no hardness; (3) distortion—no distortion of nearby skin; (4) contour—flush with surrounding skin; (5) global—no hypertrophy or keloid formation. During this dermatological assessment, evaluation at time 2 weeks was obscured by scabs, so the data for cosmetic scores was collected from evaluation at weeks 4 and 6 (see FIG. 5). FIG. 6 shows the difference between wounds sites at 6 weeks. In the top profile (relaxin treatment) the scar is invisible. In the bottom profile, the scar is visible. The wound/scar areas that received irrigation relaxin treatment were ranked higher overall than those that received only topical relaxin treatment.

Histological evaluation. A professional histopathologist evaluated the pathology of the treatments for such indications as granulation, inflammation, and necrosis. In judging granulation tissue, it is considered that early response to wound includes invasion of the site by fibroblasts, inflammatory cells, and new blood vessels. Over time, this resolves to more normal tissue as the site heals. Chronic inflammation characterized by the presence of macrophages, giant cells, lymphocytes, and PMNs indicated adverse reaction. Minor factors such as keratosis (formation of keratin layer outside the epidermis) and acanthosis (thickening of the epidermal layer) are also considered.

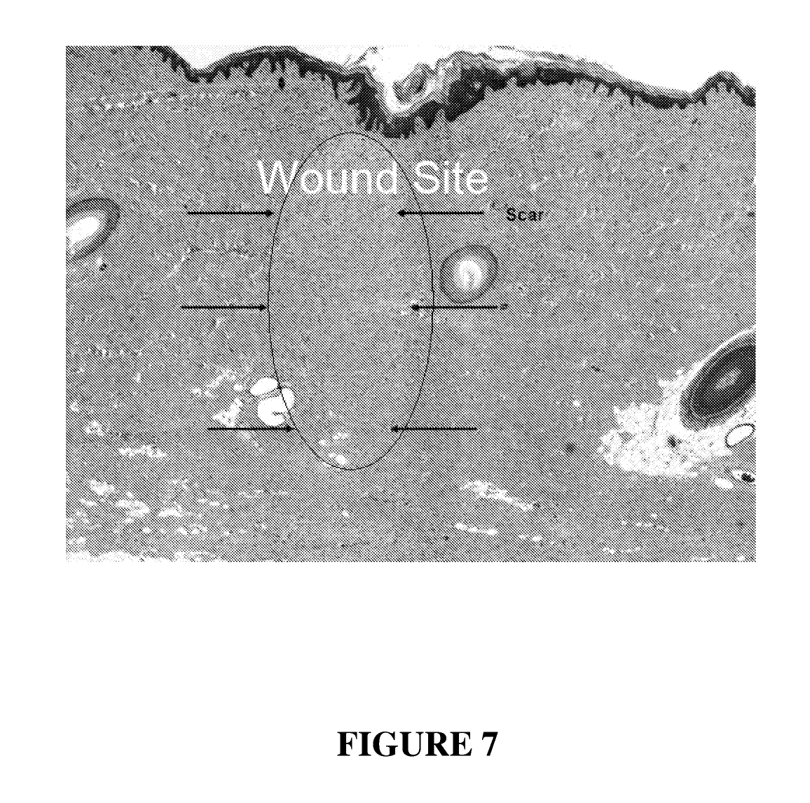

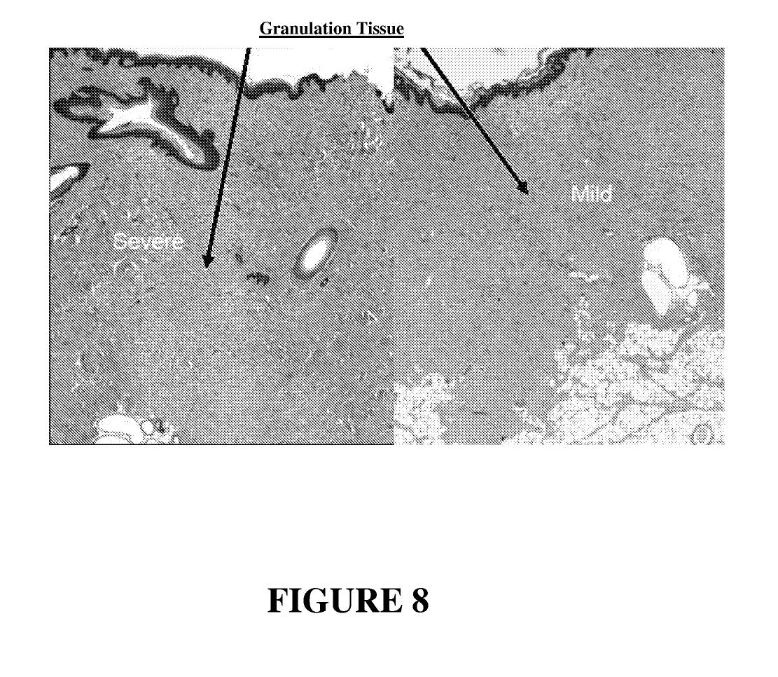

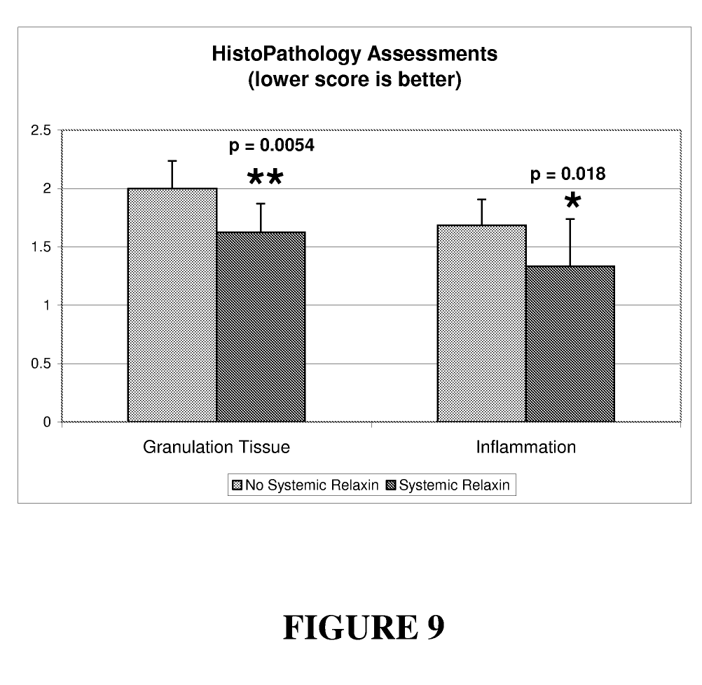

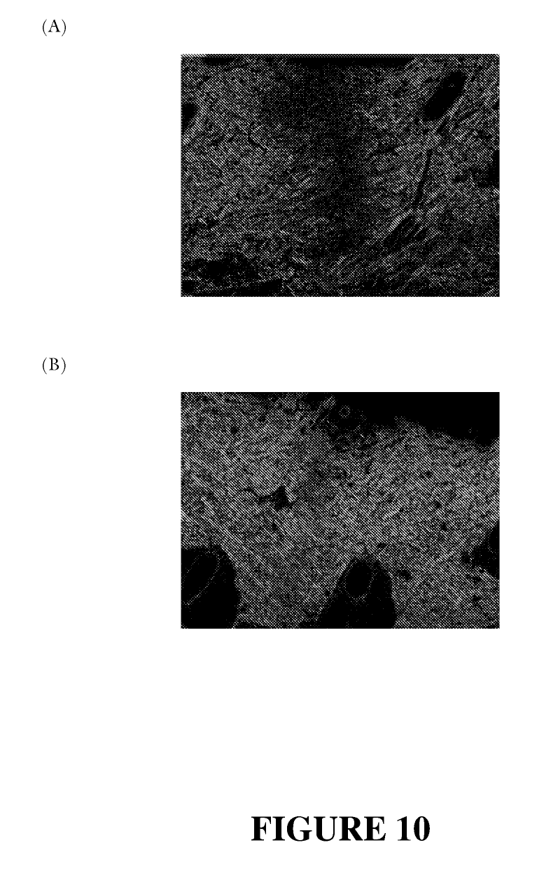

FIGS. 7 and 8 show that there are no adverse effects associated with relaxin treatment. Granulation tissue is reduced with systemic relaxin treatment (i.e., there is an indication of quicker resolution of the wound with relaxin). Finally, chronic inflammation was reduced with systemic relaxin, again showing that relaxin treatment promotes faster healing (see FIGS. 9, 10).

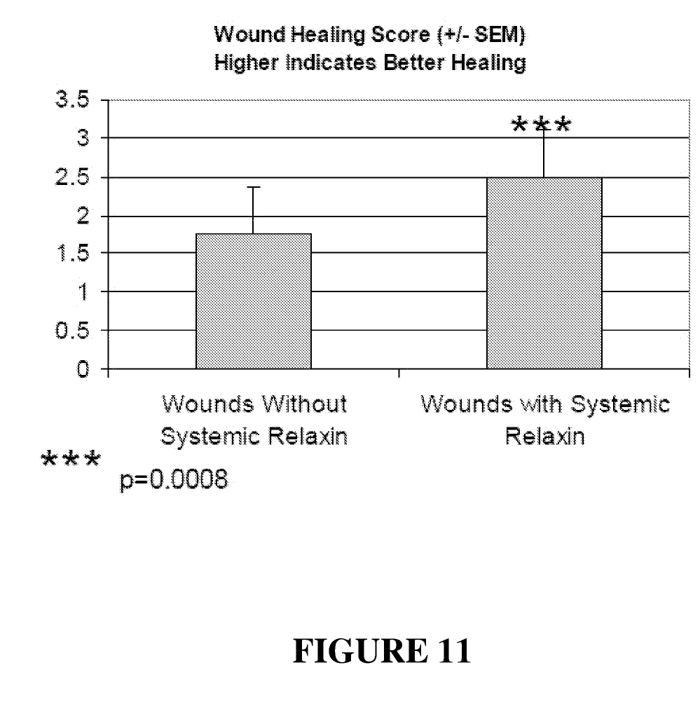

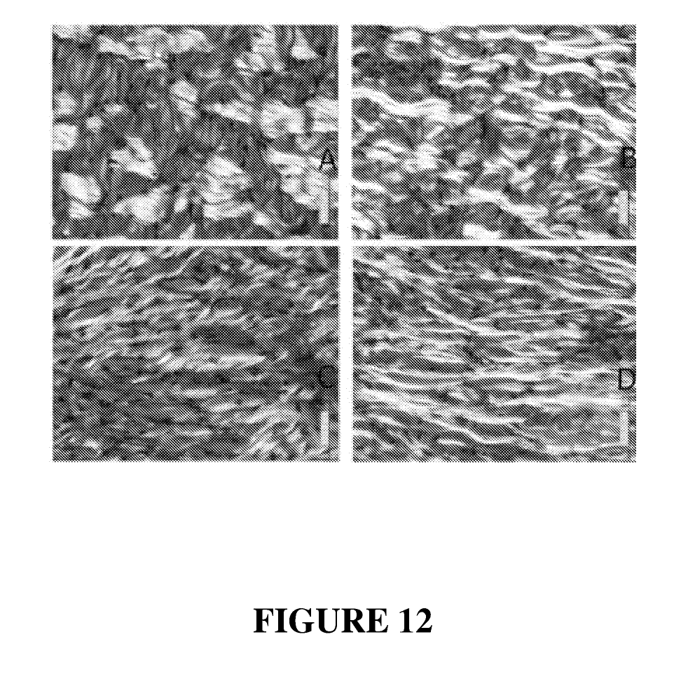

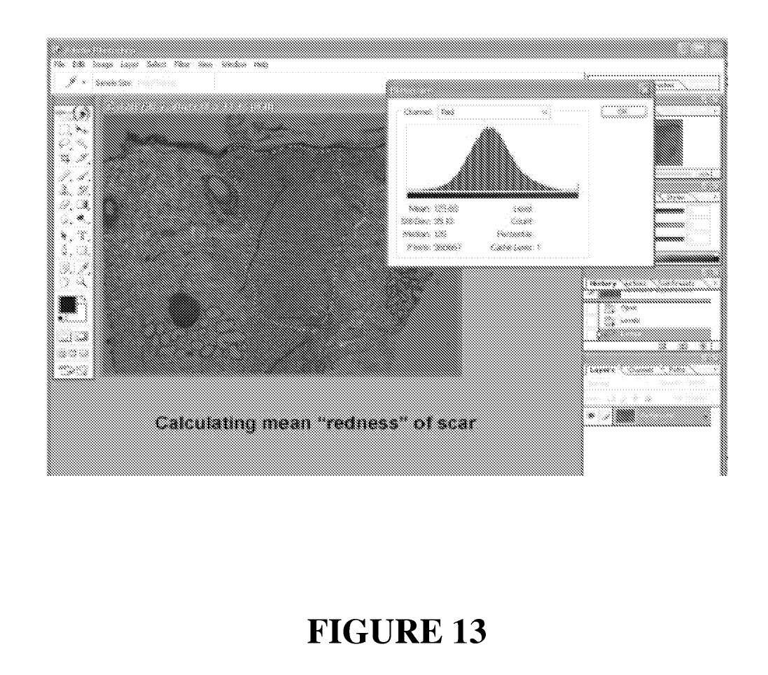

A professional histologist evaluated wound healing according to a histological evaluation that took into account a wound scoring system, collagen organization determined by red pixel counts (counted by computer program—ratio of red pixels in scar area to surrounding normal tissue indicates relative organization of collagen), and blood vessels from factor VIII staining (see FIGS. 12, 13). The wound healing score was enumerated as follows: 3 points for an interwoven arrangement of collagen fibers (best healing); 2 points for an intermediate collagen fiber arrangement and weave; and 1 point for collagen fibers arranged in parallel bundles or in one plane (worst healing). The best healing is evidenced by the interwoven arrangement of collagen fibers similar to the normal pattern but smaller in size compared to the normal dermis (see FIG. 11).

It was thus determined that no adverse effects were seen with any of relaxin treatments (i.e., irrigation or topical administration). Further, no necrosis or inflammation was associated with relaxin treatment. There are some indications that better wound healing is observed following systemic treatment with relaxin, compared to topical administration.

Findings and Conclusion. This pilot study is the first to explore the use of relaxin on animal skin. The inventor's primary goal was to establish the safety of topical relaxin on wound healing. The secondary goal of the study was to demonstrate the efficacy of relaxin in wound healing and/or scar formation. The inventor demonstrated the following: (1) Over a wide dose range (10-960 μg/kg/day), the drug showed no relevant adverse effects and was well-tolerated. (2) Relaxin produced beneficial effects by speeding up the natural wound healing process and increasing the strength of a wound site by increasing granulation tissue formation, reducing the number of blood vessels that result from angiogenesis, increasing collagen deposition, aiding in the formation of a better organized collagen matrix, and promoting the re-epithelialization of a wound.

### EXAMPLE 2

### Canine Gingiva Study

Overview of the Dog Study. Human 2 relaxin (H2 RLX) has been explored as a potential therapy in orthodontic applications due to its ability to remodel soft tissue. Previous studies in dog models have demonstrated that the application of RLX via gingival injections can speed tooth movement and prevent relapse.

The aim of this study was to determine the rate of RLX migration and extent of degradation after gingival injections. This study showed that RLX stayed close to the injection site. Employing a dog model, RLX was administered and tissue punch biopsies were collected at the site of injection and 0.5, 1 and 2 cm from the site of injection. Tissue was collected at several time points including 1, 2 and 4 hrs after injection. Protein was extracted from the gingival biopsies and analysed via ProteinChip technology (Surface Enhanced Laser Desorption/Ionization Time-of-Flight Mass Spectrometry, SELDI-TOF MS), which combines two well-established methods of solid phase chromatography and TOF-MS into an integrated platform. ProteinChip arrays were coated with anti-H2 RLX Ab, allowing the specific capture of RLX and associated breakdown/modified products.

RLX was clearly detected at all three time points, but a steady decrease in concentration was observed: at 1, 2 and 4 hrs we detected 90 nM, 78 nM and 21 nM of H2 RLX respectively. Movement of RLX through the gingiva also appears to be limited. RLX was detected 0.5 cm from the site of injection but was not detected further at the 1 cm and 2 cm marks.

Design of Drug. Gingival injections were administered (25 μg H2 RLX/100 μl, 41 μM) and tissue was collected at several positions including the site of injection and 0.5, 1 and 2 cm from the site of injection. Tissue was collected 1, 2 and 3 hrs after injection. Tissue biopsies were also collected from control animals that did not receive RLX treatment.

### Study Procedures

a) Protein Extraction and Quantitation. Frozen gingival biopsies were homogenized in extraction buffer (10 μl/mg tissue) containing 10 mM Tris-HCl, 10 mM NaCl, 0.1% TFA and protease inhibitor (complete mini, Roche). The extract was centrifuged (6000×g) for 10 min and the supernatant was collected, aliquoted and stored at −80° C. Total protein concentration was determined using the Bradford assay. Protein concentrations ranged from 1.4 mg/ml to 2.8 mg/ml, all samples were standardized to 1.4 mg/ml in extraction buffer.

b) SELDI-TOF MS analysis—Analysis of RLX treated gingiva on PG20 arrays. Anti-H2 RLX Ab (2 μl/0.25 mg/ml; Genentech) or control IgG (Ciphergen Biosystems) was applied to each spot of a PS20 array which had been pre-coated with Protein G (Ciphergen Biosystems). After antibody binding, the chip was washed once with PBS/0.5% Triton X-100 for 5 min with agitation (5 μl per spot), followed by two PBS washes. Dog gingiva extracts (3 μl/1.4 mg/ml) were applied to each spot and incubated for 4 hrs. Non-specifically bound proteins were removed by sequentially washing with PBS/0.5% Triton X-100, PBS and 1 mM HEPES, pH 7.2. The chips were air dried and 1 μl of 50% saturated sinapinic acid in 50% (v/v) acetonitrile, 0.5% trifluoroacetic acid was applied onto each spot twice, arrays were air dried between each application. Chips were subsequently analysed by SELDI-TOF MS (Ciphergen Biosystems) using the following settings, laser 220 and sensitivity 9.

c) Generation of H2 RLX Std Curve on PG20 arrays. H2 RLX (batch number 11835-89) was spiked into control dog gingiva (1.4 mg/ml) at different concentrations, including 100 nM, 50 nM, 12.5 nM, 6.25 nM, 3.125 nM and 1.56 nM and analysed as described above.

d) Assessment of H2 RLX on NP20 arrays. H2 RLX was analysed on a normal phase (NP20) array to assess purity. H2 RLX (1 μL/500 nM) was loaded onto a spot, the array was air dried and two applications of 50% SPA were applied to each spot as described above.

Findings. In order to quantitate the levels of RLX in the treated gingiva samples a H2 RLX standard curve was generated. The linear dynamic range was determined to be two orders of magnitude with a R² value of 0.9495.

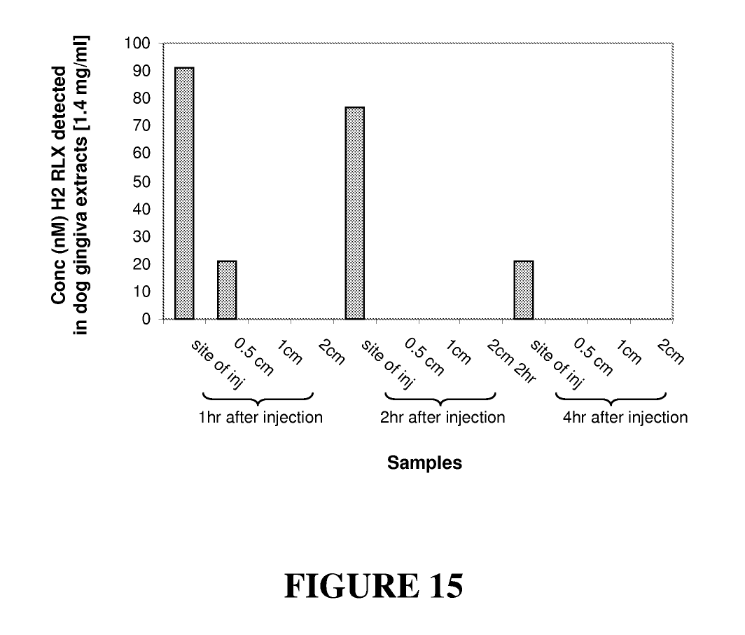

Assessment of the treated dog gingiva revealed that RLX was present at all three time points. but a steady decrease in concentration was observed; at 1, 2 and 4 hrs we detected 90 nM, 78 nM and 21 nM of H2 RLX respectively. RLX was identified to have limited migration through the gingiva. At the 1 hr time point, RLX was detected at 0.5 cm from the site of injection but was not detected at 1 cm and 2 cm from the site of injection. At the remaining time points RLX was only detected at the site of injection (see FIG. 15).

Conclusion. Thus, this study involving the relaxin treatment of dog gingiva clearly showed that relaxin stayed close to the injection site, achieving local targeting without the need to inject relaxin at various points along the scar.

### EXAMPLE 3

### Human Partially Healed Open Skin Wound Study

Partially healed open wounds have typically clotted, wherein a preliminary layer of extracellular matrix and fibrin has been laid down to bridge the clot and tissues. The goal of this study is to determine the safety of relaxin on partially healed wounds, the efficacy in human wound healing and/or scar formation, and the most efficient method of relaxin delivery to the partially healed wound to promote the desired results.

Relaxin is administered in combination with at least one other pharmaceutically active agent, which may be an NSAID or an antibiotic. In the first set of experiments, relaxin is administered via injection at the wound site. In the second set of experiments, the partially healed wound is surgically re-opened by removing a thin layer of the collagen matrix that has settled, and relaxin is administered via irrigation, which involves slowly dripping 0.5 ml of a 1.05 mg/ml relaxin solution in sodium acetate onto the wound site. In the third set of experiments, the partially healed wound is surgically re-opened by removing a thin layer of the collagen matrix that has settled, and relaxin is administered topically, which involves applying relaxin directly onto the wound site. Topical delivery contains 0.5 mL of either 0.5 mg/ml (low dose) or 2.5 mg/ml (high dose) of relaxin in a formulation consisting of 0.5 ml of either 0.5 or 2.5 mg/ml relaxin in a formulation consisting of 76.5% 20 mM sodium acetate buffer, 0.17% methylparaben, 0.03% propylparaben, 5% propylene glycol, 5% ethanol, 1% HED 250HX and 12.285% relaxin in sodium acetate buffer. In the fourth set of experiments, the partially healed wound is once again surgically re-opened by removing a thin layer of the collagen matrix that has settled, and relaxin is administered systemically via an osmotic infusion pump at 5.3 ug/kg/hr to achieve about 20 ng/ml systemic concentrations. The wound is treated twice daily for two weeks, followed by once daily treatment for three weeks.

Wound healing for each aforementioned delivery method is compared to the placebo. Visual rankings of wound pictures is conducted by neutral observers who evaluate scabs during early wound healing (days 5-7) and scars at the end of the study. Pictures of the wounds are shown to non-dermatologists for assessment. Observers are blinded to treatments and asked to assess the pictures by their own criteria, choosing and ranking the best three groups among six groups (each with eight photos), wherein three groups received relaxin and three did not.

A cosmetic score is also determined by a professional dermatologist each week. The cosmetic score has five components: (1) color—matching with surrounding skin; (2) texture—no hardness; (3) distortion—no distortion of nearby skin; (4) contour—flush with surrounding skin; (5) global—no hypertrophy or keloid formation.

Furthermore, a professional histo-pathologist evaluates the pathology of the treatments for such indications as granulation, inflammation, and necrosis. In evaluating wound healing, the histo-pathologist uses a histological evaluation that takes into account a wound scoring system, collagen organization determined by red pixel counts (counted by computer program—ratio of red pixels in scar area to surrounding normal tissue indicates relative organization of collagen), and blood vessels from factor viii staining. The best healing is evidenced by the interwoven arrangement of collagen fibers similar to the normal pattern but smaller in size compared to the normal dermis. This evaluation is important in determining whether there are adverse effects associated with any of relaxin treatments.

### EXAMPLE 4

### Human Fresh Open Skin Wound Study

Fresh open wounds and injuries on human skin are treated within two to three hours of occurrence with relaxin. The goal of this study is to determine the safety of relaxin on open wound healing in humans, the efficacy in human wound healing and/or scar formation, and the most efficient method of relaxin delivery to the open wound to promote the desired results. The injury may be a cut. which may be an incision of the epidermis, or a wound, which may be open or closed. Open wounds may include, but are not limited to, an incision, a laceration, an abrasion, a puncture wound, a penetration wound, a gunshot wound, and a stabbing wound.

Relaxin is administered in combination with at least one other pharmaceutically active agent, which may be an NSAID or an antibiotic. Delivery methods to the open wound include irrigation, topical, systemic and injection.

In the first set of experiments, relaxin is administered via irrigation, which involves slowly dripping 0.5 ml of a 1.05 mg/ml relaxin solution in sodium acetate onto the wound site. In the second set of experiments, relaxin is administered topically, which involves applying relaxin directly onto the wound site. Topical delivery contains 0.5 mL of either 0.5 mg/ml (low dose) or 2.5 mg/ml (high dose) of relaxin in a formulation consisting of 0.5 ml of either 0.5 or 2.5 mg/ml relaxin in a formulation consisting of 76.5% 20 mM sodium acetate buffer, 0.17% methylparaben, 0.03% propylparaben, 5% propylene glycol, 5% ethanol, 1% HED 250HX and 12.285% relaxin in sodium acetate buffer. In the third set of experiments, relaxin is administered systemically via an osmotic infusion pump at 5.3 ug/kg/hr to achieve about 20 ng/ml systemic concentrations. In the fourth set of experiments, relaxin is administered via injection at or in close proximity to the wound site. The fresh wound is treated twice daily for two weeks, followed by once daily treatment for three weeks.

Wound healing for each aforementioned delivery method is compared to the placebo. Visual rankings of wound pictures is conducted by neutral observers who evaluate scabs during early wound healing (days 5-7) and scars at the end of the study. Pictures of the wounds are shown to non-dermatologists for assessment. Observers are blinded to treatments and asked to assess the pictures by their own criteria, choosing and ranking the best three groups among six groups (each with eight photos), wherein three groups received relaxin and three did not.

A cosmetic score is also determined by a professional dermatologist each week. The cosmetic score has five components: (1) color—matching with surrounding skin; (2) texture—no hardness; (3) distortion—no distortion of nearby skin; (4) contour—flush with surrounding skin; (5) global—no hypertrophy or keloid formation.

Furthermore, a professional histo-pathologist evaluates the pathology of the treatments for such indications as granulation, inflammation, and necrosis. In evaluating wound healing, the histo-pathologist uses a histological evaluation that takes into account a wound scoring system, collagen organization determined by red pixel counts (counted by computer program—ratio of red pixels in scar area to surrounding normal tissue indicates relative organization of collagen), and blood vessels from factor viii staining. The best healing is evidenced by the interwoven arrangement of collagen fibers similar to the normal pattern but smaller in size compared to the normal dermis. This evaluation is important in determining whether there are adverse effects associated with any of relaxin treatments.

### EXAMPLE 5

### Human Plastic Surgery Wound Study

Open wounds that result from plastic surgeries, including both facial and body plastic surgery, are treated at the end of the surgery with relaxin to promote wound healing and to minimize scar formation. The goal of this study is to determine the safety of relaxin on wound healing, the efficacy of relaxin on wound healing and/or scar formation that result from facial or full body plastic surgery, and the most efficient method of relaxin delivery to the plastic surgery wound to promote the desired results.

Relaxin is beneficial to treatment of wounds that result from facial plastic surgery, which includes but is not limited to. rhytidectomy, blepharoplasty, rhinoplasty, otoplasty, mentoplasty, face lift, fore head lift, brow lift, facial scar revision, facial scar removal, laser surgery, skin resurfacing, wrinkle treatment, plasma skin regeneration, facial fat grafting, skin tightening, tattoo removal and hair replacement. Furthermore, relaxin is beneficial to the treatment of wounds that result from body plastic surgery, which includes but is not limited to abdominoplasty, breast reduction, breast enhancement, body lift procedures, spider vein treatment, stretch mark treatment, liposuction, excess skin removal surgery, cellulite reduction treatment, body contouring, body resurfacing and body implants.

Relaxin is administered in combination with at least one other pharmaceutically active agent, which may be an NSAID or an antibiotic. The first dose of relaxin is administered at the end of surgery via irrigation, which involves slowly dripping 0.5 ml of a 1.05 mg/ml relaxin solution in sodium acetate onto the open wound site. Subsequently, relaxin is administered topically, twice daily for two weeks, followed by once daily treatment for three weeks. Topical administration involves applying relaxin directly onto the wound site. Topical delivery contains 0.5 mL of either 0.5 mg/ml (low dose) or 2.5 mg/ml (high dose) of relaxin in a formulation consisting of 0.5 ml of either 0.5 or 2.5 mg/ml relaxin in a formulation consisting of 76.5% 20 mM sodium acetate buffer, 0.17% methylparaben, 0.03% propylparaben, 5% propylene glycol, 5% ethanol, 1% HED 250HX and 12.285% relaxin in sodium acetate buffer.

Wound healing for each aforementioned delivery method is compared to the placebo. Visual rankings of wound pictures is conducted by neutral observers who evaluate scabs during early wound healing (days 5-7) and scars at the end of the study. Pictures of the wounds are shown to non-dermatologists for assessment. Observers are blinded to treatments and asked to assess the pictures by their own criteria, choosing and ranking the best three groups among six groups (each with eight photos), wherein three groups received relaxin and three did not.

A cosmetic score is also determined by a professional dermatologist each week. The cosmetic score has five components: (1) color—matching with surrounding skin; (2) texture—no hardness; (3) distortion—no distortion of nearby skin; (4) contour—flush with surrounding skin; (5) global—no hypertrophy or keloid formation.

Furthermore, a professional histo-pathologist evaluates the pathology of the treatments for such indications as granulation, inflammation, and necrosis. In evaluating wound healing, the histo-pathologist uses a histological evaluation that takes into account a wound scoring system, collagen organization determined by red pixel counts (counted by computer program—ratio of red pixels in scar area to surrounding normal tissue indicates relative organization of collagen), and blood vessels from factor VIII staining. The best healing is evidenced by the interwoven arrangement of collagen fibers similar to the normal pattern but smaller in size compared to the normal dermis. This evaluation is important in determining whether there are adverse effects associated with any of relaxin treatments.

Various modifications and variations of the present disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are understood by those skilled in the art are intended to be within the scope of the claims.

1-30. (canceled)

31. A method of improving the cosmetic appearance of a skin wound comprising administering a pharmaceutical formulation comprising pharmaceutically active relaxin to a subject with a skin wound in an amount effective in producing a healed wound with an improved cosmetic appearance, as compared to a healed wound of an untreated subject.

32. The method of claim 31, wherein the healed wound comprises a closer color match with surrounding skin.

33. The method of claim 32, wherein the healed wound further comprises an interwoven arrangement of collagen fibers.

34. The method of claim 31, wherein the relaxin is purified, recombinant or synthetic H1, H2 or H3 human relaxin, or a relaxin agonist.

35. The method of claim 31, wherein the relaxin is administered systemically to the subject and/or topically to the skin wound.

36. The method of claim 31, wherein the pharmaceutical formulation further comprises one or both of an antibiotic and a non-steroidal anti-inflammatory drug.

37. A method for reducing scarring during healing of a skin wound comprising administering a pharmaceutical formulation comprising pharmaceutically active relaxin to a subject with a skin wound in an amount effective in producing a healed wound with reduced scarring, as compared to a healed wound of an untreated subject.

38. The method of claim 37, wherein the scar is selected from the group consisting of a keloid, a hypertrophic scar, and striae.

39. The method of claim 37, further comprising debridement or removal of pre-existing scar tissue.

40. The method of claim 37, wherein the relaxin is purified, recombinant or synthetic H1, H2 or H3 human relaxin, or a relaxin agonist.

41. The method of claim 37, wherein the relaxin is administered systemically to the subject and/or topically to the skin wound.

42. The method of claim 37, wherein the pharmaceutical formulation further comprises one or both of an antibiotic and a non-steroidal anti-inflammatory drug.

43. The method of claim 37, wherein the subject is a human subject with impaired healing capability.

44. A method of promoting healing of a wound comprising administering a pharmaceutical formulation comprising pharmaceutically active relaxin to a subject with a wound in an amount effective in promoting healing of the wound.

45. The method of claim 44, wherein the wound is derived from plastic surgery.

46. The method of claim 45, wherein the plastic surgery is facial or full body plastic surgery selected from the group consisting of rhytidectomy, blepharoplasty, rhinoplasty, otoplasty, mentoplasty, face lift, fore head lift, brow lift, facial scar revision, facial scar removal, laser surgery, skin resurfacing, wrinkle treatment, plasma skin regeneration, facial fat grafting, skin tightening, tattoo removal, hair replacement, abdominoplasty, breast reduction, breast enhancement, body lift procedures, spider vein treatment, stretch mark treatment, liposuction, excess skin removal surgery, cellulite reduction treatment, body contouring, body resurfacing and body implants.

47. The method of claim 44, wherein the relaxin is purified, recombinant or synthetic H1, H2 or H3 human relaxin, or a relaxin agonist.

48. The method of claim 44, wherein the relaxin is administered systemically to the subject and/or topically to the skin wound.

49. The method of claim 44, wherein the pharmaceutical formulation further comprises one or both of an antibiotic and a non-steroidal anti-inflammatory drug.

50. The method of claim 44, wherein the subject is a human subject with impaired healing capability.
